(19) United States
(12) Patent Application Publication
(10) Pub. No.: US 2023/0398343 A1
(43) Pub. Date: Dec. 14, 2023

(54) Catheter Connectors for ECG-based Catheter Positioning Systems

(71) Applicant: NAVI MEDICAL TECHNOLOGIES PTY LTD, Box Hill, Victoria (AU)

(72) Inventors: Alexander NEWTON, Box Hill, Victoria (AU); Shing Yue SHEUNG, Box Hill, Victoria (AU); Christiane THEDA, Box Hill, Victoria (AU); Mubin YOUSUF, Box Hill, Victoria (AU)

(21) Appl. No.: 18/035,666

(22) PCT Filed: Nov. 6, 2021
(86) PCT No.: PCT/IB2021/060293

§ 371 Date: May 5, 2023

(30) Foreign Application Priority Data
Nov. 6, 2020 (AU) 2020904044

(51) Int. Cl. A61M 39/10; A61B 5/283; A61B 5/367

(52) U.S. Cl. CPC A61M 39/10; A61B 5/283; A61B 5/367; A61B 2562/227

(57) ABSTRACT

A catheter connector, comprising a body defining a lumen extending between first and second ends of the body, a cable port in a sidewall of the body between the first and second ends; and an electrode arranged internally of the sidewall and the cable port to provide electrically conductive contact between saline received in the lumen and an end of an electrical cable received in the cable port.

### FIELD

The present invention relates generally to catheter connectors for use with electrocardiogram (ECG)-based catheter tip positioning systems.

### BACKGROUND

Catheter connectors (or adapters), such as those used in ECG-based catheter tip positioning systems, often comprise a body that fluidly connects a catheter (such as a central venous catheter (CVC), a peripherally inserted central catheter (PICC), or an umbilical venous catheter (UVC)) to a saline-flushing syringe, and a cable that electrically connects ECG electronics of the ECG-based catheter tip positioning system to an electrically conductive saline column inside the catheter. Optimal tip positions of the catheter can include positions near the inferior vena cava (IVC) or superior vena cava (SVC).

Migration of catheter tips after positioning is not uncommon. Post-positioning chest X-rays are routinely performed to confirm the position of the tip of the catheter, and to detect tip migration or malposition. This process is time consuming, exposes patients to radiation, and does not provide real time feedback on catheter tip position.

Existing ECG catheter connectors suffer from several drawbacks which are barriers to ECG-guided catheter tip positioning systems being routinely used as alternatives to chest X-rays for routine post-positioning catheter tip migration checks or position confirmations.

Some existing ECG catheter connectors have a fixed cable which connects to the ECG electronics. The fixed cable is inconvenient when not in use, and only adds unnecessarily to the tangle of tubing and wires permanently connected to patients (sometimes referred to as “Spaghetti Syndrome”).

Other existing ECG catheter connectors have an external cable connection terminal which removably connects a cable to the ECG electronics. The external cable connection terminal poses electrical shock and contamination risks when exposed after the cable is disconnected. In addition, existing removably connectable cables suffer from a number of a performance and reliability issues, including low electrical current load, poor signal integrity, high electrical signal noise and interference, and open gaps and creep after connection.

In view of this background, there is an unmet need for improved catheter connectors for use with ECG-based catheter tip positioning systems.

### SUMMARY

According to the present invention, there is provided a catheter connector, comprising:

a body defining a lumen extending between first and second ends of the body;

a cable port in a sidewall of the body between the first and second ends; and

an electrode arranged internally of the sidewall and the cable port to provide electrically conductive contact between saline received in the lumen and an end of an electrical cable received in the cable port.

The first and second ends of the body may removably connect, directly or indirectly, to a syringe and a catheter via luer connectors.

The end of the electrical cable may removably connect to the cable port by a removable connector.

The removable connector may comprise a bayonet connector, a luer connector, a magnetic connector, or a radio-jack connector.

The cable port may have a self-sealing closure that keeps the cable port sealed when the end of the electrical cable is disconnected.

The body and the cable port may be arranged in a T-shape or a Y-shape.

The electrical cable may removably connect the catheter connector to ECG electronics.

The electrical cable may have a plug end to removably connect to the cable port, and a socket end to removably connect to the ECG electronics.

The electrical cable may further comprise an inline remote controller for the ECG electronics between the plug end and the socket end.

The catheter connector and the electrical cable may be provided as a kit of parts for use with the ECG electronics.

The present invention also provides a method, comprising:

monitoring positioning of a tip of a catheter within a patient using ECG electronics connected to the catheter via a catheter connector when connected to the ECG electronics by an electrical cable;

disconnecting the electrical cable from the catheter connector after positioning of the tip of the catheter within the patient;

reconnecting the electrical cable to the catheter connector to confirm positioning of the tip of catheter within the patient using the ECG electronics.

### BRIEF DESCRIPTION OF DRAWINGS

Embodiments of the invention will now be described by way of example only with reference to the accompanying drawings, in which:

FIG. 1 is a side rendering of a catheter connector according to one embodiment of the present invention;

FIGS. 2 and 3 are perspective renderings of another embodiment of the catheter connector;

FIG. 4 is a perspective rendering of the catheter connector connected to a syringe and a catheter;

FIG. 5 is a schematic diagram of the catheter connector connected to ECG electronics of an ECG-based catheter tip positioning system;

FIG. 6 is a perspective rendering of a plug end of an electrical cable for the catheter connector;

FIG. 7 is a perspective rendering of a socket end of the electrical cable;

FIG. 8 is a perspective rendering of an inline remote controller of the electrical cable;

FIG. 9 is a schematic diagram of one embodiment of the electrical cable being used with a guidewire adapter of a guidewire-assisted catheter tip positioning system; and

FIG. 10 is a schematic diagram of another embodiment of the electrical cable having the guidewire adapter at one end.

### DESCRIPTION OF EMBODIMENTS

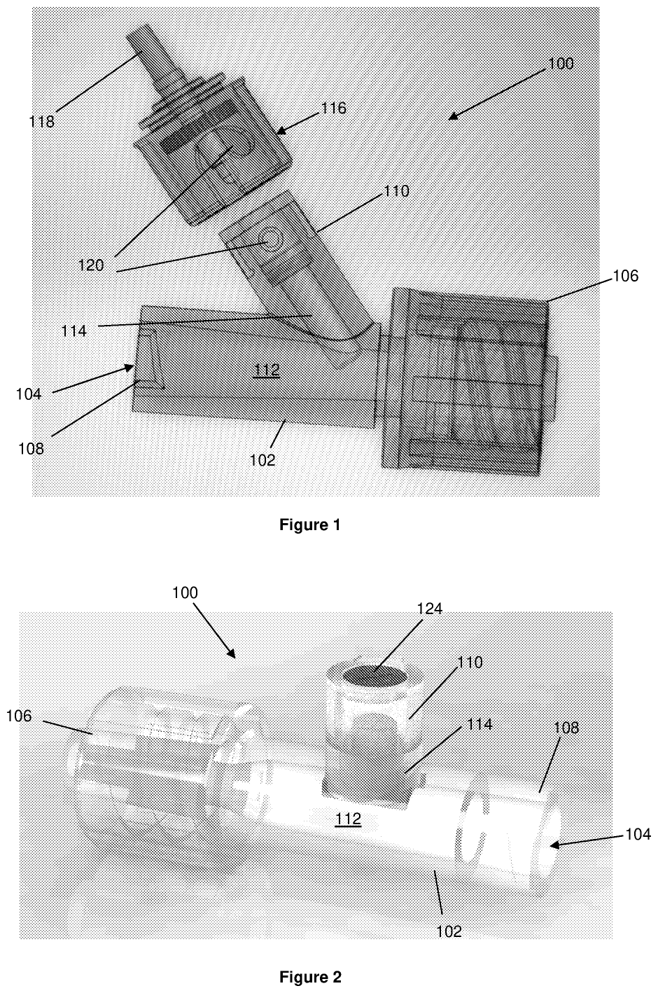

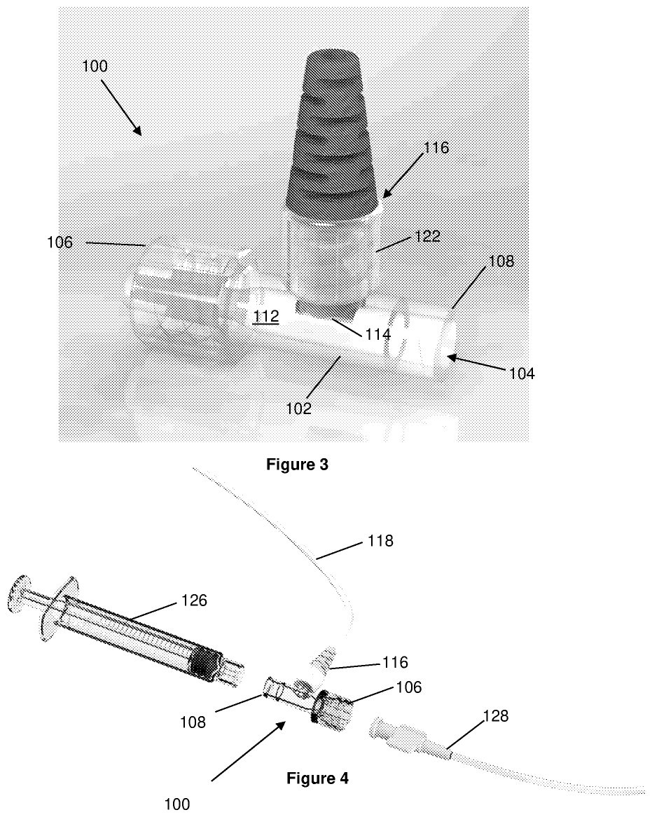

Referring to FIGS. 1 to 3, a catheter connector 100 according to an embodiment of the present invention may comprise a body 102 defining a lumen 104 extending between first and second ends 106, 108 of the body 102. A cable port 110 may be provided in a sidewall 112 of the body 102 between the first and second ends 106, 108. An electrode 114 may be arranged internally of the sidewall 112 and the cable port 110 to provide electrically conductive contact between saline received in the lumen 104 and an end 116 of an electrical cable 118 received in the cable port 110.

The body 102 and the cable port 110 may be arranged in an overall Y-shape as shown in FIG. 1, or a T-shape as shown in FIG. 2. The end 116 of the electrical cable 118 may removably connect to the cable port 110 by a removable connector. The removable connector may, for example, comprise a bayonet connector, a luer connector, a magnetic connector, or a radio-jack connector. Other alternative or equivalent types of removable electrical connections may also be used.

As shown in FIG. 1, in one embodiment, the end 116 of the electrical cable 118 may removably connect to the cable port 110 via a bayonet connector 120, such as a BNC or Bayonet Neil-Concelman mating connector pair. In another embodiment shown in FIG. 2, the end 116 of the electrical cable 118 may removably connect to the cable port 110 via a luer lock connector 122. The luer lock connector 122 may comprise a tapered male connector tip surrounded by an internally threaded collar on the end 116 of the electrical cable 118 providing a screw fit, in addition to a friction fitting, with a corresponding female connector part on the cable port 110.

As shown in FIG. 2, the cable port 110 may have a self-sealing closure 124 that keeps the cable port 110 sealed when the end 116 of the electrical cable 118 is disconnected. The self-sealing closure 124 may, for example, comprise a luer-activated split-septum.

Referring to FIG. 4, the first and second ends 106, 108 of the body 102 may removably connect, directly or indirectly, to a syringe 124 and a catheter 126 via luer connectors. For example, the first end 106 of the body 102 may comprise a female luer connector that directly removably connects to a male luer connector of the syringe 124. The second end 108 of the body 102 may comprise a male luer connector that directly removably connects to a female luer connector of the catheter 124. In other embodiments, the first and second ends 106, 108 of the body 102 may indirectly removably connect to the syringe 124 and the catheter 126 via other components in a catheter stack (not shown), such as a three-way stop cock and a needleless connector. In use, saline may be flushed from the syringe 124 through the lumen 104 of the body 102 of the catheter connector 100 into the catheter 126.

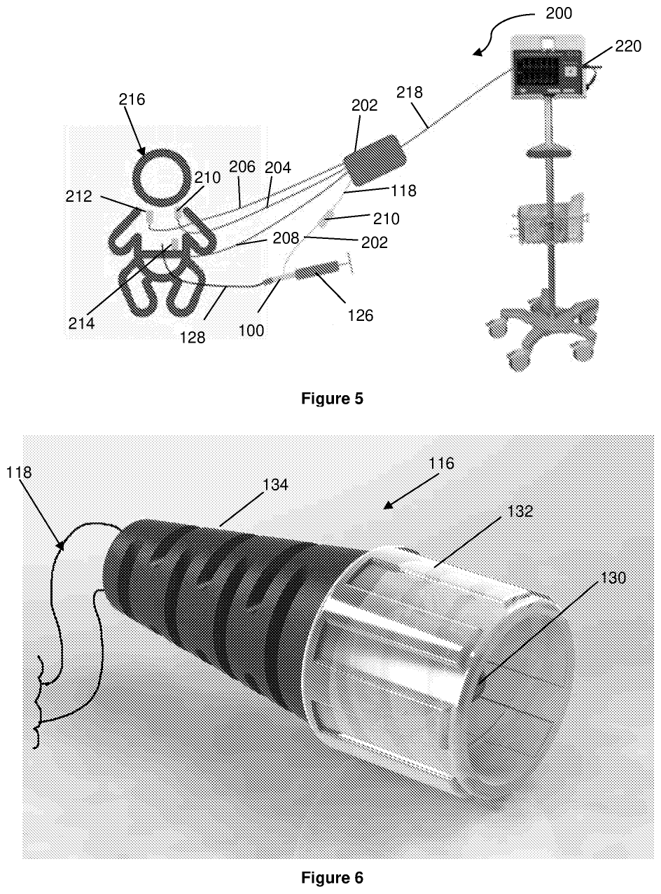

Referring to FIG. 5, the electrical cable 118 may removably connect the catheter connector 100 to ECG electronics 200. The ECG electronics 200 may comprise an ECG acquisition module 202 (or interface, or patient lead connector) which connects leads 204, 206, 208 to surface electrodes 210, 212, 214 on the left-arm, right arm, and left leg (or left flank) of a patient 216. The ECG acquisition module 202 may be removably connected to the catheter connector 100 by the electrical cable 118, and by a fixed cable 218 to an ECG-based catheter tip positioning system 220, such as the present applicant's Neonav® system described in WO 2019/075529 which is hereby incorporated by reference in its entirety.

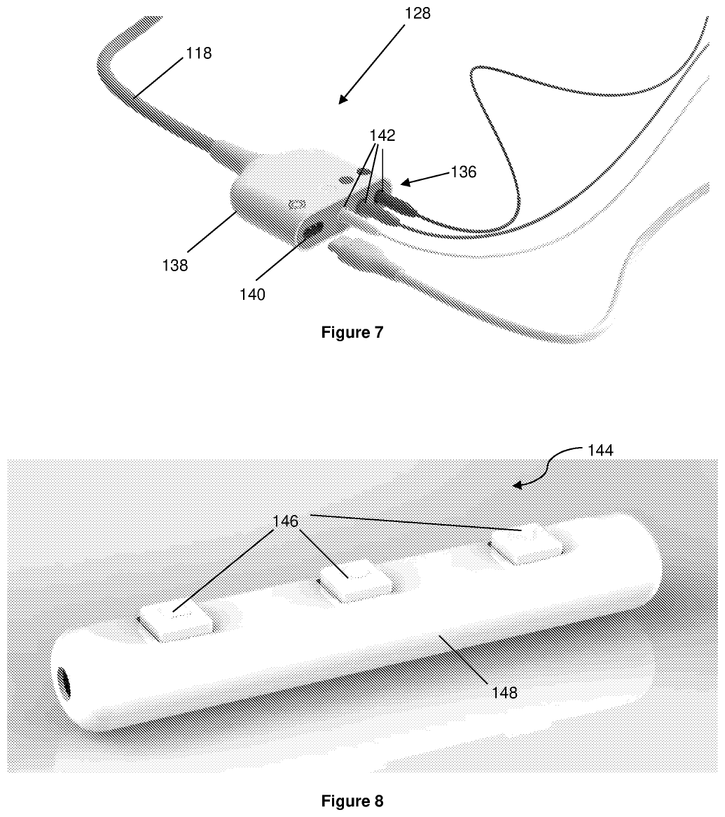

Referring to FIGS. 6 and 7, the electrical cable 118 may have a plug end 116 to removably connect to the cable port 110, and a socket end 128 to removably connect to the ECG electronics 200. The plug end 116 may refer to a first end of the electrical cable 118 that is fitted with and in electrical connection with an electrical plug connector 130. As shown in FIG. 6, the plug end 116 may comprise an electrical plug connector 130 in an insulative housing 132. The insulative housing 132 may comprise a resilient collar 134. The electrical plug connector 130 may, for example, comprise a male electrode 130 that electrically connects to a female electrode 114 arranged internally of the sidewall 112 and the cable port 110. Other alternative or equivalent types of electrical plug connectors may also be used.

In embodiments where the plug end 116 of the electrical cable 118 is removably connected to the cable port 110 via a bayonet connector 120 or a luer lock connector 122, the male electrode may have a spring (or pogo pin) (not shown) to reduce the risk of overtightening of the connection which can lead to damage to the male/female electrodes 130, 114, and to allow for slight gaps due to manufacturing tolerances. The spring may be actuated at around 50-75% when the male and female electrodes 130, 114 are engaged and the luer/bayonet connection is secured. For the bayonet connector 120, a J-notches or tracks may be provided in an end of the male electrode 130 to engage complementary pins or studs on the female electrode 114 that may be received in and engage with the notch or tracks for a secure connection.

The socket end 128 may refer to a second end of the electrical cable 118 that is fitted with and in electrical connection with electrical socket connectors 136. As shown in FIG. 7, the socket end 128 may comprise electrical socket connectors 138 in an insulative housing 140. The electrical socket connectors 138 may comprise, for example, a DIN connector and a female radio jack socket that electrically connect to electrical plug connectors of the ECG electronics 200. Other alternative or equivalent types of electrical socket connectors may also be used.

Referring to FIG. 8, the electrical cable 118 may further comprise an inline remote controller 142 for the ECG electronics 200 between the plug end 116 and the socket end 128. The inline remote controller 142 may comprise push, press, tap, swipe or touch controls 144 on a printed circuit board (not shown) in an insulative housing 146. The inline remote controller 142 may, for example, be configured with remote controls for the ECG electronics 130 including forward, back, new reading, undo, increase/decrease recorded insertion depth, resize ECG waveform viewing axis, impedance variable element, etc. Other alternative or equivalent controls or functions may also be used.

The components of the catheter connector 100 and the electrical cable 118 that are not electrically conductive may be made from electrically insulative materials, such as plastic materials. The plastic materials may be selected to provide ease of cleaning and sterilisation, and to provide transparency to observe saline flow and positive connection of components.

The electrically conductive components of the catheter connector 100 and the electrical cable 118 may be made of metals. The metals may be selected for compatibility with medical environments and procedures. For example, the electrode 114 arranged internally of the sidewall 112 and the cable port 110 may be made from austenitic stainless steel to allow the catheter connector 100 to be in placed under an active MRI scanner.

Electromagnetic shielding materials or structures may be provided for the electrically conductive components of the catheter connector 100 and the electrical cable 118 to reduce electromagnetic interference with the ECG electronics 200 and other electromagnetic interference emitting equipment in the environment. For example, metallic layers, tapes or films may be used to form electromagnetic shielding sheathes, meshes or cages around electrically conductive components, such as around the electrode 114 arranged internally of the sidewall 112 and the cable port 110 of the catheter connector 100, and around the electrical plug connector 130 of the plug end 116 of the electrical cable 118. The electromagnetic shield may also be connected to the reference electrode on the patient to further reduce the effect of electromagnetic interference.

The connector components of the catheter connector 100 and the electrical cable 118 may be coloured-coded, standard-sized and non-standard sized to guide correct connection to standard-sized connectors of the catheter 124, syringe 126 and ECG electronics 200, and to avoid or minimise misconnection. For example, the luer connector used to removably connect the plug end 116 of the electrical cable 118 to the cable port 110 of the catheter connector 100 may be non-standard sized compared to standard-sized luer connectors of the catheter 124 and the syringe 126.

The catheter connector 100 and the electrical cable 118 may be provided as a kit of parts for use with the ECG electronics 200.

The catheter connector 110 may be used in a method which starts by monitoring positioning of a tip of a catheter 124 within a patient 216 using ECG electronics 200 connected to the catheter 124 via the catheter connector 100 when connected to the ECG electronics 200 by the electrical cable 118.

Next, the electrical cable 118 may be disconnected from the catheter connector 100 after positioning of the tip of the catheter 124 within the patient 216.

The method may end by reconnecting the electrical cable 118 to the catheter connector 100 to confirm positioning of the tip of catheter 124 within the patient 216 using the ECG electronics 200.

It will be appreciated that, if desired, the electrical cable 118 may alternatively be left connected to the catheter connector 100 after initial positioning of the tip of the catheter 124 so that reconnection of the electrical cable 118 before subsequent position confirmation is unnecessary. Further or alternatively, the electrical cable 118 may be left connected to the catheter connector 100 after initial positioning of the tip of the catheter 124 for continuous monitoring of positioning by the ECG-based catheter tip positioning system 220.

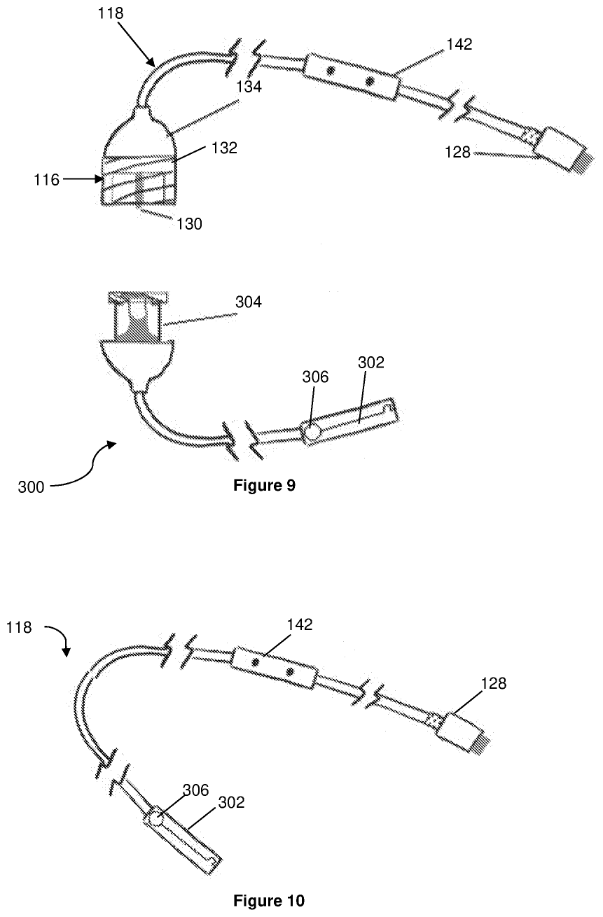

Referring to FIG. 9, the electrical cable 118 may be adapted for cooperation with a guidewire-assisted catheter location system by a guidewire adapter 300 to indirectly removably connect the plug end 116 of the electrical cable 118 to a guidewire 302 of the guidewire-assisted catheter location system. One end of the guidewire adapter 300 may comprise a female luer connector 304 for cooperation with the male luer lock connector 122 of the plug end 116, and the other end may comprise a snap lock guidewire connector 306 for snap locking to the guidewire 302.

In another embodiment illustrated in FIG. 10, the guidewire adapter 300 may be directly substituted for the plug end 116 of the electrical cable 118 to directly removably connect the guidewire 302 to the electrical cable 118.

Examples of use of the catheter connector 100 and the electrical cable 118 have been provided above for use with ECG-based catheter tip positioning systems. The invention is not limited to the examples that have just been given. Those skilled in the art will appreciate that the examples may be reproduced for use with other types of medical electronics without difficulty, and with similar success, by substituting any of the generically or specifically described components or steps mentioned anywhere in this specification for those actually used in the preceding examples.

Embodiments of the present invention provide a catheter connector and an electrical cable that are both generally and specifically useful for ECG-based catheter tip positioning systems.

For the purpose of this specification, the word “comprising” means “including but not limited to,” and the word “comprises” has a corresponding meaning.

The above embodiments have been described by way of example only and modifications are possible within the scope of the claims that follow.

1. A catheter connector, comprising:
a body defining a lumen extending between first and second ends of the body;
a cable port in a sidewall of the body between the first and second ends; and
an electrode arranged internally of the sidewall and the cable port to provide electrically conductive contact between saline received in the lumen and an end of an electrical cable received in the cable port.

2. The catheter connector of claim 1, wherein the first and second ends of the body removably connect, directly or indirectly, to a syringe and a catheter via luer connectors.

3. The catheter connector of claim 1, wherein the end of the electrical cable removably connects to the cable port by a removable connector.

4. The catheter connector of claim 3, wherein the removable connector comprises a bayonet connector, a luer connector, a magnetic connector, or a radio-jack connector.

5. The catheter connector of claim 1, wherein the cable port has a self-sealing closure that keeps the cable port sealed when the end of the electrical cable is disconnected.

6. The catheter connector of claim 1, wherein the body and the cable port are arranged in a T-shape or a Y-shape.

7. The catheter connector of claim 1, wherein the electrical cable removably connects the catheter connector to ECG electronics.

8. The catheter connector of claim 7, wherein the electrical cable has a plug end to removably connect to the cable port, and a socket end to removably connect to the ECG electronics.

9. The catheter connector of claim 8, wherein the electrical cable further comprises an inline remote controller for the ECG electronics between the plug end and the socket end.

10. The catheter connector of claim 1, wherein the catheter connector and the electrical cable are provided as a kit of parts for use with the ECG electronics.

11. A method, comprising:
monitoring positioning of a tip of a catheter within a patient using ECG electronics connected to the catheter via a catheter connector when removably connected to the ECG electronics via an electrical cable;
disconnecting the electrical cable from the catheter connector after positioning of the tip of the catheter within the patient;
reconnecting the electrical cable to the catheter connector to confirm positioning of the tip of catheter within the patient using the ECG electronics.
